(19) United States
(12) Patent Application Publication
(10) Pub. No.: US 2023/0036561 A1
(43) Pub. Date: Feb. 2, 2023

(54) OSTEOINDUCTIVE AND OSTEOCONDUCTIVE IMPLANT OR BIOACTIVE SCAFFOLD SURFACE AND METHOD FOR PRODUCING SUCH A SURFACE

(71) Applicants: Diego Pedreira DE OLIVEIRA, São Carlos - SP (BR); Diego Pedreira DE OLIVEIRA, São Carlos - SP (BR); Luis Felipe Sverzut BARONI, São Carlos - SP (BR)

(72) Inventor: Diego Pedreira DE OLIVEIRA, São Carlos - SP (BR)

(73) Assignees: Lucas De Almeida MAGALHÃES, São Paulo- SP (BR); Diego Pedreira DE OLIVEIRA, São Carlos - SP (BR); Luis Felipe Sverzut BARONI, São Carlos - SP (BR)

(21) Appl. No.: 17/788,570

(22) PCT Filed: Nov. 17, 2020
(86) PCT No.: PCT/BR2020/050473

§ 371 Date: Jun. 23, 2022

(30) Foreign Application Priority Data
Dec. 23, 2019 (BR) BR 10 20190277750

(51) Int. Cl. A61F 2/30; C23F 1/26; C23F 1/38

(52) U.S. Cl. CPC A61F 2/30771; A61F 2/3094; C23F 1/26; C23F 1/38; A61F 2002/30838; A61F 2002/3084; A61F 2002/3093

(57) ABSTRACT

A method for constructing a three-dimensional multi-scale surface to obtain controlled and improved physical and chemical configurations to promote the integration of orthopedic and/or dental implants, to human and/or animal tissues, in different shapes and geometries in a versatile manner, and can be applied to all types of metals, metal alloys and/or ceramic compounds. This method includes the modification at the macroscopic level of the roughness, with an objective of promoting the mechanical interlocking of the implant, followed by the modification of the surface for the formation of microtopography, then the microtopography is changed to obtain a nanotopography with characteristics that optimize cellular metabolic responses related to attraction, adhesion, spreading, proliferation and cell growth, in addition to phenotypic and genotypic inductions in undifferentiated cells and in osteoblast lineage, responsible for mineralization and bone neoformation. As a result, the interface between implant and bone is improved.

### FIELD

The bioactive, osteoinductive and osteoconductive surface of implants or scaffolds and method of producing thereof object of this invention is applied to the surfaces of orthopedic and/or dental bone surgical implant elements, for humans and/or animals, which are presented in different shapes and designs in a versatile way, and can be applied to all types of metallic alloys, polymeric and ceramic materials to constitute an adaptable surface in nanosized three-dimensional scales.

### BACKGROUND

Humanity follows an aging and growth trend, with this, the number of dental and orthopedic surgeries has been growing consistently year after year. Commonly, people replace their teeth and bony joints using implants and prosthetic solutions.

Following this trend, successful implants at immediate or late loads require properties that can induce well-developed osseointegration. Osseointegration is related to intimate contact between the implant/bone observed by the resolution of microscopy with optical light. The term is important, however, the way to define the best osseointegration is not consensual and depends on both the properties of the host tissue and the implant surface.

Osseointegration can be described as a functional connection between the bone and the surface material under demand-for-load conditions. When no progressive movement is verified, the implant has direct contact with the bone, minimizing any local or systemic adverse biological response.

For example, an implant with a polished, therefore smooth, surface can easily lead to the appearance of relative micro-movements between implant and bone, then triggering inflammatory events detrimental to osseointegration.

In addition, the surfaces can be adapted to provide specific micro and nano environments to induce and drive tissue regeneration associated with osteoinductive and osteoconductive mechanisms. Essentially, surface modification aims to provide chemical and physical substrates to stimulate stem cells, mesenchymal cells, pre-osteoblasts, osteoblasts, that is, specified and non-specific cells, to induce mineralization stimulation fundamental for biological stability and for the osseointegration process.

Osseointegration can be established by stimulating or catalyzing events that induce osteoinduction and osteoconduction. Osteoinduction aims to activate non-specific cells, that is, immature cells, mesenchymal stem cells or pre-osteoblasts to configure active cells in the osseointegration process, the osteoblasts. Osteoconduction is related to the process of mineralization and conduction of bone tissue on the micro and nano topography previously formed on the surface of the implants. These events can contribute and accelerate a well-established osseointegration, without movements between the implants and the bone.

The strategy to develop implantable devices exploiting tissue regeneration as a method for osseointegration is based on producing osteoinductive and osteoconductive surface properties that modulate the implant osseointegration process, producing a high-quality implant/bone interface.

The industry has developed surface technologies to improve bone implant connections (osseointegration). The production of roughness and geometries on a macro scale proved to be an appropriate strategy to favor the mechanical stability of the implants. Therefore, mechanical interlocking is of fundamental importance to avoid relative micro-movements between implant/bone and, therefore, to avoid any inflammatory stimulation derived from this process. At a subsequent stage, biological stability is required to modulate and favor interface formation and contribute to the long life of these implants. To improve the response and interface of implants to bones and tissues, surface modification on a micro and nano scale is a key element.

Thus, the synergistic effect between mechanical interlocking or primary stability and the constitution of the interface leads to a well-established osseointegration phenomenon. Based on this scientific knowledge, the surfaces developed by these processes constitute a fundamental substrate to provide an environment that leads to biological responses. By controlling this sequence of events, the biological stability of the implants can be optimized, and the interface well developed.

Observing the heterogeneity of requirements for biomedical solutions and especially the trends in the development of producing processes, the processes of modification and treatment of versatile, adjustable and customizable surface for complex projects and geometries have technological importance for the next generations of implant developments, using additive producing processes in the several industrial sectors. This invention was developed to meet these requirements.

Currently, the accepted classification for dimensional levels establishes that macroscale comprises structural characteristics above 10 μm, while microscale is from 1 to 10 μm, submicro is from 0.1 μm to 1.0 μm, and nanoscales comprise structures below 100 nm.

Based on this classification, the surface presented can be characterized as a multiscale surface, providing macro-rugosity associated with micro, submicro and nanotopography, similar to the coral structure associated with the sponge effect, that is, with micro-coral-like topography and the ability to incorporate ions and molecules to the surface, which is composed of micro and nanotopography that can function as a scaffold with nano dimensions, therefore, with high effective area and with the ability to absorb, adsorb and incorporate bio-ions and molecules, thus favoring the adhesion of cells mediated by bio-molecules.

Enriched with phosphorus-based compounds and other elements existing in the bone tissue, the titanium oxide layer is modified to obtain nanostructured surface with bio-ions, which allows the regulation from gene adhesion to expression of human osteoblasts. Micro and nanostructured topography can be applied to previously rough or smooth orthopedic and dental implants. If the previous layer requires a rougher condition produced both by means of additive processes, such as titanium plasma spraying, and by means of subtractive processes, such as the blasting of particles or spheres, the treatments can be perfectly adjustable for any prior macro-scale design, geometry and surface shape.

There is a relationship between the process of cleaning the surface of implants using different acidic means and the formation of roughness. The existence of chlorides, fluorides and sulfates on the implant surface is generally related to the surface acid packaging and does not favor the biological processes related to osseointegration.

There are some patent documents that describe implants and implant surface treatments, as well as surgical methods for implant adhesion, however, none of these documents anticipates the method and the surface proposed herein, where it can be applied to any metallic implant, not only dental, but also orthopedic and cardiological, also provides for macroscale modification, not only by subtractive methods (blasting, surface attack), but also by additive methods (TPS, PVD) and produces controlled surface in all scales (macro, micro and nano), with characteristics in weak geometry and sponge properties. Controlled topography in micro and nanoscales is capable of increasing cellular attraction and adhesion, controlling cellular gene expression dynamics, and providing bioactive, osteoinductive, osteoconductive, and antimicrobial properties to surfaces. Among these documents, the following can be highlighted:

Patent document PI 0510301-0, “IMPLANTES DE METAL DE INDUÇÃO DE OSSO PARA UM CORPO VIVO E PROCESSO DE PRODUÇÃO DOS MESMOS” describes metal material implants on which a layer of bioactive material, more specifically, hydroxyapatite, is applied to give the implants stimulating properties for bone growth. This invention differs from the cited document since produces osteoconductive and osteoinductive properties by modifying the implant surface, without the need to add material to the surface;

Patent document US 2017/0360532 “TITANIUM NANO-SCALE ETCHING ON AN IMPLANT SURFACE” describes a surface treatment for dental implants to obtain nanoscale pores on the implant surface only by acid etching;

Patent document US20160220740 “BIOLOGICALLY ACTIVE IMPLANTS” describes the application of coating of polymeric material on metallic material with subsequent impregnation of anti-infection agents. The method of this invention is based on surface modification and not material addition by coating;

Patent document US20120219599 “OSTEOGENIC PROMOTING IMPLANTS AND METHODS OF inducing BONE GROWTH” teaches the osteoconductive properties derive from the nature of the scaffold material and the osteoinductive properties of the molecule impregnated in the material of the scaffold. This invention achieves these properties by modifying the implant surface by physicochemical methods;

Patent document US20130189323 “ANTIBACTERIAL AND OSTEINDUCTIVE IMPLANT COATING, METHOD OF PRODUCING SUCH COATING, AND IMPLANT COATED WITH SAME” teaches the antibacterial and osteoconductive properties are conferred to the implant through the addition of a copper-doped calcium phosphate coating. The method of this invention performs surface treatment and obtains these characteristics by modifying the topology of the surface without adding coating;

Patent document US20140363392 “OSTEOINDUCTIVE COATINGS FOR DENTAL IMPLANTS” teaches the osteoconductive properties are conferred to the implant through the addition of a polymeric coating. The method of this invention performs surface treatment and obtains these characteristics by modifying the topology of the surface without adding coating;

US2017/0354504 “PROTEIN DELIVERY WITH POROUS METALLIC STRUCTURE” describes a porous matrix implant that is loaded with bone graft material having osteoconductive properties impregnated with a protein having osteoinductive properties. This invention confers such properties to the implant by modifying the topology and surface chemistry, without the need for use of bone graft material and/or protein impregnation;

Patent document US20170319750 “COMPOSITE MATRICES DESIGNED FOR ENHANCED BONE REPAIR” describes a biocompatible implant composed of polymer matrix and ceramic material, with subsequent addition of coating. This method applies to metallic materials, has no addition of ceramic material or coating;

US20150072017 “CARRIER MATERIALS FOR PROTEIN DELIVERY” teaches osteoconductive properties are added to the implant by a mineral component coating that serves as the basis for loading a protein with osteoinductive properties. This invention produces osteoconductive and osteoinductive properties by modifying the implant surface, without the need to add an osteoinductive coating or molecule;

Patent document US20130178946 “COMPOSITE DEVICE THAT COMBINES POROUS METAL AND BONE STIMULATION” teaches the implant is composed of porous metal material on which a layer of resorbable material is applied that confers on the implant osteoconductive and osteoinductive properties. This invention produces osteoconductive and osteoinductive properties by modifying the implant surface, without the need to add resorbable material or osteoinductive molecule;

Patent document WO201727426 “IMPROVED CERAMIC AND/OR GLASS MATERIALS AND RELATED METHOD” describes a chemical treatment on ceramic material to generate osteoconductive properties. This invention is intended for metallic materials;

Patent document WO201328735 “MEDICAL DEVICE FOR BONE IMPLANT AND METHOD FOR PRODUCING SUCH DEVICE” describes an implant with osteoconductive and osteoinductive properties, a metallic implant covered by a substance capable of generating osteoinductive and osteoconductive properties. This invention is intended for metallic implants and produces osteoconductive and osteoinductive properties by modifying the implant surface, without the need for the addition of material.

Other than that reported in WO201328735, wherein the technology addresses the producing of holes in the surface to be loaded with therapeutic agents. The surface invention proposed herein comprises microporosity and nanostructured topographic sponge effect to incorporate biological agents. The scientific and technological concept of surface design aims to avoid the vulnerability of mechanical failure induced by the promotion of stress concentration on the surface. Fatigue failure susceptibility is avoided by surface chemical treatments associated with TPS coatings for orthopedic implants and on shaded surfaces for dental implants. The mechanism of osteoinduction on the surface of implants stimulates processes of differentiation of undifferentiated cells, mesenchymal stem cells, for example, in osteoblastic cell line suitable for the process of bone mineralization. How to properly combine and adjust the surface at macro, micro and nano levels mentioned herein is what provides the described mechanisms.

Patent document WO201775613 “MATRIX FOR ENHANCED DELIVERY OF OSTEOCONDUCTIVE MOLECULES IN BONE REPAIR” describes an implant with osteoconductive and osteoinductive properties, composed of a polymer matrix with addition of ceramic material acting as a bone growth stimulating agent. This invention is intended for metallic implants and produces osteoconductive and osteoinductive properties by modifying the implant surface, without the need for the addition of material;

Patent document WO201703461 “OSTEOCONDUCTIVE AND OSTEOINDUCTIVE IMPLANT FOR AUGMENTATION, STABILIZATION, OR DEFECT RECONSTRUCTION” describes an implant with osteoconductive and osteoinductive properties composed of a polymeric matrix with impregnation of a bone growth stimulating agent. This invention is intended for metallic implants and produces osteoconductive and osteoinductive properties by modifying the implant surface, without the need for the addition of material; and

Patent document CN107376018 “STRONTIUM-CONTAINING BIOLOGICAL MATERIAL AND ITS PREPARATION METHOD AND THE ONE APPLICATION” describes a strontium-containing material, which can be used to impart osteoconductive and osteoinductive properties to the implant.

This invention produces osteoconductive and osteoinductive properties by modifying the implant surface, without the need for material addition.

### SUMMARY

This disclosure relates to a new method for constructing a three-dimensional multi-scale surface, the surface obtained and applications thereof, which is a combination of surface modification processes to obtain controlled and optimized physical and chemical configurations to promote the integration of orthopedic and/or dental implants, to human and/or animal tissues, in different shapes and geometries, in a versatile manner, and can be applied to all types of metals, metal alloys and/or ceramic compounds and/or polymers. This method comprises the optional modification at the macroscopic level of the roughness, with the objective of promoting the mechanical interlocking of the implant, followed by the modification of the surface for the formation of microtopography; then, the microtopography is changed in a nanoscale to obtain a nanotopography with characteristics that optimize cellular responses related to attraction, adhesion, spreading, proliferation and cell growth, in addition to phenotypic and genotypic inductions in cells of the osteoblast lineage, responsible for mineralization and bone neoformation. With this, the interface between implant and bone is improved, as well as the efficiency of the implant osseointegration process compared to other surface environments.

The bioactive, osteoinductive and osteoconductive surface of implants or scaffolds and method of producing thereof object of this invention is applied to the surfaces of orthopedic and/or dental bone surgical implant elements, for humans and/or animals, which are presented in different shapes and designs in a versatile way, and can be applied to all types of metallic alloys, polymeric and ceramic materials to constitute an adaptable surface in nanosized three-dimensional scales.

It is an objective of the bioactive, osteoinductive and osteoconductive surface of implants or scaffolds and method of producing thereof, to offer to the orthopedic and/or dental surgical implants, and other tissues market, a method of producing and applying a surface, or surface modifications to the implant elements to obtain optimized configurations of substrates, improving cellular metabolic responses related to gene adhesion, growth and expression, then promoting bone connection of the implant.

### BRIEF DESCRIPTION OF THE FIGURES

In the following, reference is made to the figures accompanying this specification, for a better understanding and illustration thereof, where it is seen:

FIG. 1 shows the flowchart of the processes involved in modifying the bioactive, osteoinductive and osteoconductive surface of implants or scaffolds and method of producing thereof, object of this invention;

FIG. 2 provides a photograph of the surface under analysis with MEV at low and high magnifications and can be observed nano characteristics of 10 to 300 nm, surface modification with acid treatment in phosphate-containing medium combined with alkaline treatment for titanium alloys. In addition, increased surface area and hydrophilic behavior culminated in increased surface energy;

FIG. 3 shows an example of another titanium alloy subjected to surface modification processing to transmit the micro to nanotopographic evidence, in two increases;

FIG. 4 shows evidence of trimodal surface of the titanium alloy, processed by a plastic deformation sequence, with macro, micro and nanostructured pores; three levels of modification can at least be observed;

FIG. 5 shows the attractiveness of the titanium surface before and after being immersed in the treatment for a few hours, as in this example, after four hours in cell culture medium solution;

FIG. 6 shows the attraction of bio-ions in a few hours of treatment immersion, highlighting the gene expression of SP7 transcription factor on smooth, macro and micro surfaces with nanotopography (nano, in the graph);

FIG. 7 shows in vitro mineralization, osteoinductive effect, on the bioactive, osteoinductive and osteoconductive surface of implants or scaffolds;

FIG. 8 shows macro and nano-treated surfaces for the osseointegration process, on the bioactive, osteoinductive and osteoconductive surface of implants or scaffolds;

FIG. 9 shows the surface that stimulates spraying and communication between cells, as well as the formation of biogenesis-induced structures.

### DETAILED DESCRIPTION OF THE EMBODIMENTS

The bioactive, osteoinductive and osteoconductive surface of implants or scaffolds, object of this invention, is a three-dimensional engineering surface carried out on a body, comprising a physically and chemically controlled and organized topography, containing a macroscopic topography, with structures larger than 10 μm, on which a microscopic and submicroscopic topography is superimposed, with structures between 10 μm and 100 nm, on which a nanometric topography is superimposed, with structures between 1 and 100 nm, further comprising nano characteristics and structures in a fractal dimension, similar to the structure of a coral of the seabed (biomimetization), on which ions, particles or molecules can be adhered.

The bioactive, osteoinductive and osteoconductive surface of implants or scaffolds, object of this invention, as defined above, has bioactive properties, wherein the bioactive properties are related to, but not limited to, bone tissue.

Further the invention presents sponge properties, which favor the incorporation of ions, particles or molecules and exhibit hydrophilic properties, forming contact angles with water below 90 degrees, tending to 0.

The bioactive, osteoinductive and osteoconductive surface of implants or scaffolds, object of this invention, is a surface where the body is made of a metal or metal alloy where the macroscopic topography is based on the surface area, ranging from 1000% to 50% of effective increase of area after procedures of conformation, deposition or subtraction of surface material, with a roughness R_{z} and S_{z} controlled between 0 to 1000 micrometers (μm).

The surface has a microscopic topography that includes the investigation of characteristics around 0.1 to 100 micrometers, including submicrometer topography.

The controlled surface parameters can be pointed with roughness with arithmetic mean deviation (linear or spatial) around 0 to 100 μm; parameters R_{z} and S_{z} in the form of 0.1 to 100 μm; Sₛₖ from 1.0 to −1.0, where the tendency to zero is preferable; and Sₖᵤ from 0 to 10.0, preferably, tending to 3.0.

The surface has a nanoscopic topography built on the microscopic topography, presents structures with nanometric dimensions in the form of threads, fibers, pores about 10 nm thick with aspect ratio shape between 10 and 1000.

The surface has physical shapes that can be described with fractal dimension parameter, with porous formation at different scales of dimension increase.

The surface has different levels of dimensions that provide a substrate suitable for intimal contact of cells. The porous formation of these structures may be from 50 μm to 1.0 μm. Then, with greater increase, there are pores from 1.0 to 0.1 μm and, in turn, with greater increase in the microscope, structures below 100 nm are found and characterized, thus, the effective surface area presents a high increase in relation to the initial surface without treatment, which gives it a thermodynamically metastable surface energy when compared to the surface without treatment, which causes the property of incorporating ions that are part, but are not limited to the group of biological ions (K⁺, ca²⁺, sr²⁺, Mg²⁺, PO₄²⁻) and of adhering particles that are part, but are not limited to, the group of calcium phosphates with strontium incorporations, in addition to adhering molecules that are part, but are not limited to the group of cell adhesive biomolecules, such as osteopontin, actins, integrins and others, thus providing a significant improvement in the bone connection of orthopedic and/or dental surgical implants.

According to the characteristics described above, the bioactive, osteoinductive and osteoconductive surface of implants or scaffolds, object of this invention, presents the advantages of being a surface with properties of attraction and adhesion of bio-ions and biomolecules, in particular, comprising, but not limited to, the ions K⁺, Ca²⁺, Sr²⁺, Mg²⁺, PO₄²⁻, which are capable of improving metabolic activities and also providing substrate to the molecules of the group, including, but not limited to, osteopontin, actins, integrins and bioactive molecules for specific effects; which has increased properties of attraction and cell adhesion, where the cells are part of, but not limited to, the group consisting of human mesenchymal stem cells, osteoblastic cells, platelets and monocytes; which dynamically controls the expression of the cellular gene, where the genes are part of, but not limited to, the group of genes that control the osteoinduction process, osteoconduction process and osteogenic process. The sponge-effect surface allows the incorporation of substances that also exert anti-infective functions.

Thus, the implant applying such a surface has bioactive, osteoinductive and osteoconductive properties.

The method of obtaining the bioactive, osteoinductive and osteoconductive surface of implants or scaffolds, object of this invention, consists of treating the implant surface by any type of macrotopographic processing to obtain the macro-structured surface. Then, the surface is cleaned and prepared for topographic micromodifications caused by controlled chemical and/or electrochemical treatment. Then, a new chemical and/or electrochemical treatment is applied to produce nano characteristics in fractal dimension, conferring structure with sponge effect in micro and nano scale enriched with bio-ions, adjustable for complex geometries and designs. Treatment with chemical and/or electrochemical micro-modification, followed by treatment with controlled alkaline medium, results in micro and nanotopography, which comprises characteristics that promote sensory contact with cells in the phyllopodial dimension, providing interaction in the nanoenvironment with cells adhered and fixed to the surface.

The ability and property of micro and nanosponge is due to the attractiveness with biochemical ions and biomolecules, comprising ions capable of improving metabolic activities, in addition to providing the substrate with the ability to incorporate and dope P, Na, Sr, K, Mg and Ca, then the immobilization and anchoring of molecules such as osteopontin, actins, integrins. Then, the surface undergoes immersion in deionized water and controlled drying, obtaining a surface with metastable surface energy and reactive with the physiological medium, with increased cell adhesion and bioactive effects, osteoconductors and osteoinductives.

The macroscale modification can be achieved using physical and metallurgical methods. Additive and subtractive processing is employed in the production of implant surfaces to provide mechanical interlocking, avoiding micromovements between the implant and the bone. Extractive processes such as sandblasting, cold blasting (in some cases), and additive procedures such as metal plasma spraying (TPS) can provide changes at macro scales.

By inducing the formation of structures or roughness around 100 μm, primary stability can be achieved on the macroscopic scale and movements can be reduced. Micromodifications are carried out using chemical and/or electrochemical treatments to increase the effective surface area and provide the doping of compounds with phosphorus in the reconstitution of the oxide, thus stimulating cell adhesion. Surface phosphorus enrichment is important to improve surface chemical condition and provide adhesion of cells under favorable energetic conditions. The cells preferably adhere to the surface, having different surface energy from equilibrium, being hydrophilic, metastable and with phosphorus addition.

In addition, nano topography provides a suitable substrate for interacting with the cell membrane, which can favor cell proliferation and communication between cells, a fundamental role to provide connections to the vascularization and irrigation process of adjacent biological systems. Osteocytes differentiated from osteoblastic cells are responsible for converting into vascularized tissues.

Thus, long-term tertiary stability can be favored and established.

The surface of the final product, after immersion in solutions containing bio-ions, can be enriched appropriately. The ionic attractiveness of this surface can be measured after immersion in body solutions or culture media. High enrichment with elements such as P, Ca, Na, Mg, Sr and K was found, as well as high cell adhesion and spraying.

Negative surface energy does not provide physicochemical barrier to cell adhesion, cell proliferation and spraying events. The described invention may achieve the most negative adhesion surface interfacial free energy of 35 mJ·m⁻².

These conditions demonstrated a high number of cells adhered to the surface and altered morphologies, sparse, spread and circumferential, capable of inducing osteoblastic differentiation, gene expression and events related to bone tissue mineralization.

In summary, the macro roughness, surface energy, micro and nanotopography obtained can change the shape of the nucleus of the cells, reason to stimulate gene expression of certain proteins. Bone formation depends on a cascade of events in the biological field. For this reason, these developed multiscale surfaces play a key role in cell-substrate interaction. A high-performance interface can be achieved and osseointegration improved.

Considering the biomechanical complexity followed by biological events, the reported surface was carefully sized to provide unique characteristics in the substrate that constitutes a favorable environment for cells to build the high-performance interface. An increased success rate can be stimulated, just as a reduced cure time can be achieved, both with these conditions.

### EXAMPLES

Surface modification with acid treatment in phosphate-containing medium combined with alkaline treatment for titanium alloys are shown in FIG. 2, in which the surface of a screw-shaped implant has been completely and homogeneously coated.

Surface construction follows a sequence of procedures to provide macro, micro, and nano modifications that are controlled to be constructed on the surface. Roughness and undulation can be applied on a macro scale used to improve mechanical stability, then coated by chemical and/or electrochemical treatments to provide the substrates present in the sequence of examples.

FIG. 4 shows the titanium alloy, processed by a plastic deformation sequence. After preparation and metallurgical processing, this material was subjected to the processing sequence, described herein, for surface modification. Using acid treatments, topographic changes can be caused due to the formation susceptible to the acid extraction process. This morphology can still be improved with the alkaline treatment already described herein. These combinations provide a structured bimodal surface, capable of improving the contact area of the implant and bone, from macro to nanoscale. By subtracting with acid some regions composed of soluble metal phases, the surface was adapted to improve all levels of connection between the implant/bone.

Additionally, these surfaces can provide a sponge containing structure that mimics a micro and nanometric coral reef, which functions as substrate and reservoir for ions that cooperate in biological activities related to the metabolic reactions of cells. This technology is inspired by the composite bone itself and its complexly organized structure, containing porous structures that also function as a reservoir for biochemical reactions. According to these considerations, the described surfaces at multiscale levels from macro to nano have been modulated to provide the suitable substrate for bone integration to the implant.

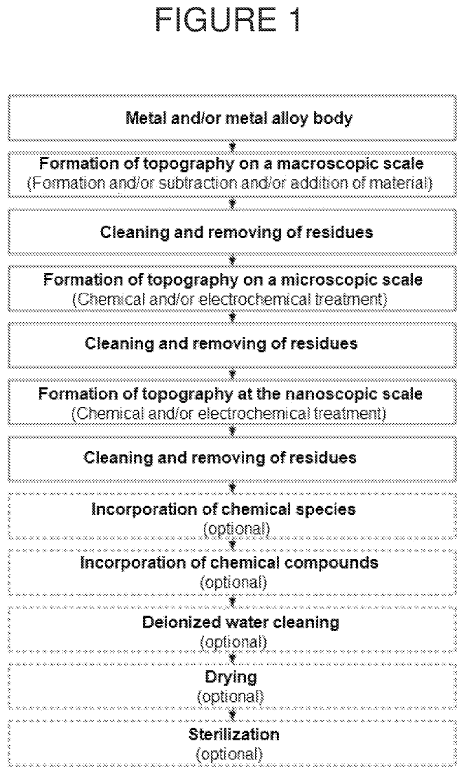

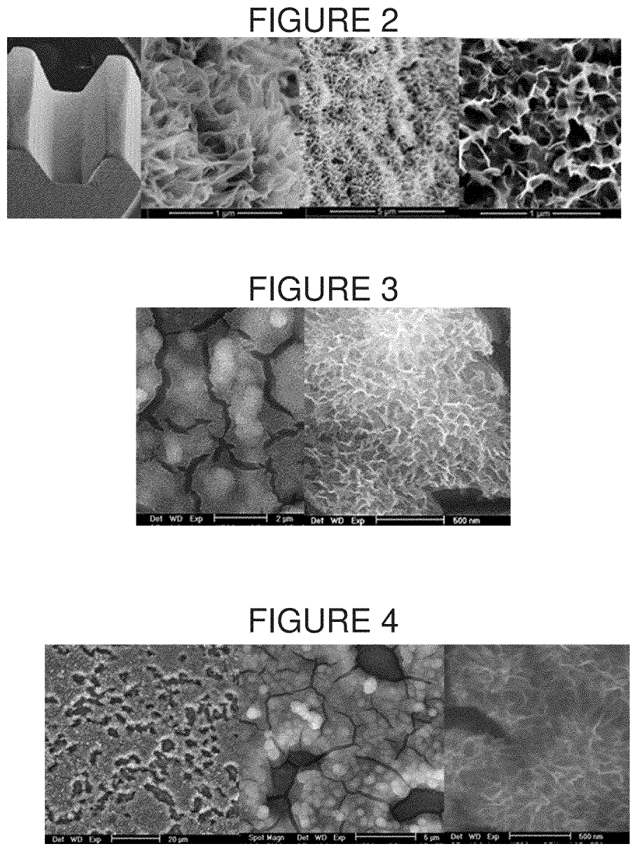

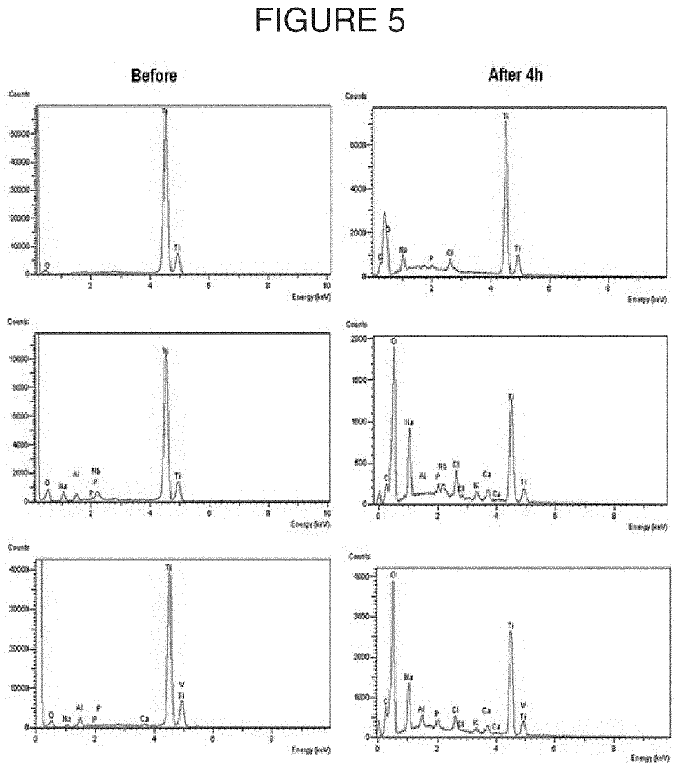

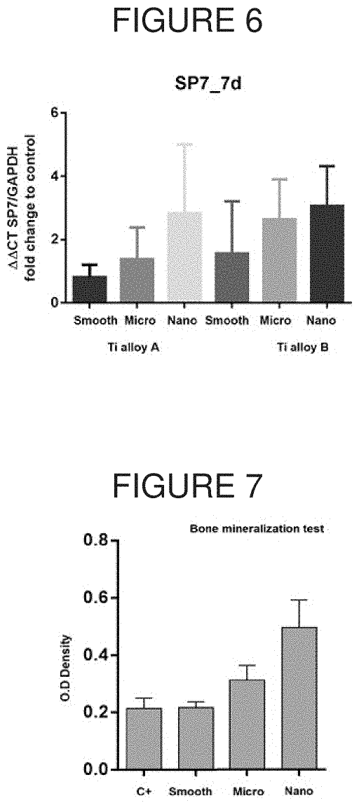

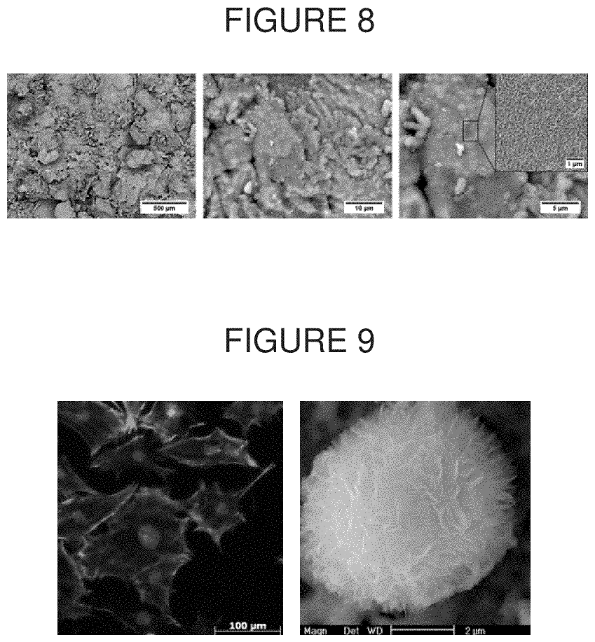

By providing this set of macro- to nano-engineered characteristics, the surfaces amplify the processes linked to bone mineralization in contact with pluripotent cells, observed in FIGS. 6 and 7, higher concentrations of mineralization on topographically altered micro and nano surfaces, compared to the material under smooth conditions. SP7 encodes genes specific to transcription processes during the differentiation process. This behavior, associated with mineralization in FIG. 1, can demonstrate and evidence this important mechanism found in this surface developed in multiscale. In addition, osteoinduction refers to the efficiency with which the substrate induces the osteoblastic differentiation process, as well as the bone formation process. The expression of this gene is linked to both processes.

These results highlight the importance of surface properties to induce osteoblastic lineage differentiation associated with mineralization events and demonstrate osteoinductive and osteoconductive conditions of the surface with the claimed characteristics.

Innovative behaviors for these types of surface constructions can be found and the proposed processing combination demonstrated attractive forces and high adhesion for both polar and nonpolar fluid types. This behavior can provide a strong adhesion capacity, attracting a wide range of proteins in a versatile way. This synergistic effect culminates in high surface energy and better cell adhesion to this amphiphilic surface.

Evaluating the gene expression induced by the aforementioned treatment, there is a positive regulation of bone-related proteins, indicating the potential of the surface to modulate the behavior from osteoblasts to mesenchymal stem cells.

Finally, this innovation is based entirely on the combination of procedures capable of improving biological responses in synthetic material applied to the material/tissue interface of the body.

The high forces of attraction and high adhesion for both types of fluid, polar and nonpolar, constituting amphiphilic characteristics, which can favor a strong adhesion capacity, attracting a wide range of proteins in a versatile way, and providing a substrate for cell adhesion and spreading, FIG. 9 (left), in which cells labeled with green staining and the nucleus in blue can be observed, demonstrating the adhesion force present, caused by the surface, associated with the effect of cell communication and interaction, fundamental for the development, transport of substances and irrigation of bone tissues.

Indeed, associated with this biological behavior, surface technology stimulates bioactivity, promoting the formation of calcium phosphate-based crystals, FIG. 9 (right).

This synergistic effect culminates in a reactive surface, with unstable surface energy (negative or positive), which favors cell adhesion, in addition to high cell spreading and communication between the ends of the cells, precursors of tissue irrigation to be completely formed.

Finally, this innovation is based entirely on the combination of procedures that produce a complex and organized structure at macro, micro and nanoscale levels, capable of providing cells with the stimulation that allow inducing undifferentiated cells in osteoblastic cells and synergistically can favor processes to improve biological responses in synthetic material applied to the material/tissue interface of the body.

In this way, the bioactive, osteoinductive and osteoconductive surface of implants or scaffolds and method of producing thereof, object of this invention, as described above, presents a new and unique configuration that configures great advantages in relation to the implant elements and methods of obtaining them currently used and found in the market. Among these advantages, we can mention: the fact that it can be applied to any metallic implant, not only dental, but also orthopedic, cardiological, among others; the fact that it provides for macroscale modification, not only by subtractive methods (sandblasting, surface attack), but also by additive methods (TPS, PVD, among others); the fact that it performs chemical and/or electrochemical treatment for surface modification on a micro scale, not with sulfuric and hydrochloric acid mixture solution; the fact that it performs chemical and/or electrochemical treatment for surface modification on a nano scale without the use of hydrogen peroxide; the fact of producing a controlled surface at all scales (macro, micro and nano); the fact of producing a nanoscale surface with fractal characteristics and sponge properties, with capacity for attraction and absorption of bio-ions, bio-molecules and substances that can be impregnated to the surface, as well as doping of bio-ions; the fact of producing a surface with controlled topography at the nanoscale capable of increasing cell attraction and adhesion; the fact of producing a surface with controlled topography at the nanoscale, capable of controlling the dynamics of cellular gene expression; and the fact of producing a surface with controlled topography in nanoscale with osteoinductive and osteoconductive properties.

Thus, due to the configuration and operation characteristics described above, it can be clearly noted that the bioactive, osteoinductive and osteoconductive surface of implants or scaffolds and method of producing thereof is a product and obtaining method new to the state of the art, which has unprecedented conditions of innovation, inventive step and industrialization, which make it deserve the privilege of patent.

1-6. (canceled)

7. Bioactive, osteoinductive and osteoconductive surface of implants or scaffolds, wherein the surface is a three-dimensional engineering surface carried out to a body, the surface of implants or scaffolds comprising: a physically and chemically controlled and organized topography containing a macroscopic, smooth topography and/or having macrometric surface structures, on which a microscopic topography is superimposed; micrometric and/or submicrometric surface structures on which a nanoscopic topography is superimposed; nanoscopic surface structures, containing nano characteristics and structures in fractal dimension, and a surface on which any chemical species and/or chemical compounds can be incorporated.

8. The bioactive, osteoinductive and osteoconductive surface of implants or scaffolds and the surface of claim 7, wherein said body is made of a metal or metal alloy, where the macroscopic topography is obtained by conformation, and/or subtraction, and/or deposition of material with a roughness R_{z} and S_{z}, between 1000 to 0 micrometers (μm).

9. The bioactive, osteoinductive and osteoconductive surface of implants or scaffolds and the surface of claim 7, further comprising a microscopic topography including submicroscopic topography, with surface structures between 0 to 100 μm, with controlled surface parameters: roughness with parameters (R a and S a) between 0 to 100 μm; parameters (Rz, Sz) between 0 to 100 μm; Ssk from 1.0 to −1.0; and Sku from 0 to 10; and Sm between 0 and 250 μm.

10. The bioactive, osteoinductive and osteoconductive surface of implants or scaffolds and the surface of claim 7, further comprising a nanoscopic topography with surface structures between 0 to 100 nm, with controlled surface parameters: roughness with parameters Ra and Sa between 0 to 1 μm; parameters Rz, and Sz between 0 to 1 μm; Ssk from 1.0 to −1.0; and Sku from 0 to 10; where the surface has a fractal dimension (Df) between 2 and 3, with structures of sizes less than 100 nm, regardless of the aspect ratio and shape of the structures, and with isotropic configuration.

11. The bioactive, osteoinductive and osteoconductive surface of implants or scaffolds and the surface of claim 7, further comprising bioactive properties related to bone tissue, hydrophilic properties with dynamic contact angles below 90 degrees, properties of high attraction and adhesion of ions, comprising ions K+, Ca2+, Sr2+, Mg2+, PO42− and molecules, comprising osteopontin, actins, integrins, osteocalcin and bioactive molecules; properties of high cell attraction and adhesion, in which the cells are part of the group consisting of multipotent cells, human mesenchymal stem cells, pre-osteoblastic cells, osteoblastic cells, osteocytes, osteoclasts, fibroblasts, red blood cells, leukocytes, platelets and monocytes; control of dynamics of cellular gene expression, in which the genes are part of the group of genes that control the osteoinduction process, osteoconduction process and osteogenic process, with osteoconductive and osteoinductive properties.

12. A method of making the bioactive, osteoinductive and osteoconductive surface of implants or scaffolds and the surface of claim 7, comprising: surface treatment of bodies comprising metals and/or metal alloys by processing to obtain a physically and chemically controlled and organized three-dimensional engineering surface, in macro, micro and nanoscale, through the following actions: surface modification for the formation of a topography on a macroscopic scale by conformation and/or subtraction processing and/or addition of material in the surface of the body; cleaning the surface for the removal of any type of undesirable residue; modifying the surface for the formation of a topography on a microscopic and/or submicroscopic scale through chemical treatment and/or electrochemical treatment; cleaning the surface for the removal of any undesirable residue; modifying the surface for the formation of a topography on a nanoscopic scale carried out through chemical treatment and/or electrochemical treatment; cleaning surface cleaned for the removal of any undesirable residue, chemical species are incorporated into the surface; incorporating chemical compounds to the surface; washing and cleaning the surface in deionized water, drying the surface and sterilizing the surface.
